(19) United States
(12) Patent Application Publication
(10) Pub. No.: US 2016/0271293 A1
(43) Pub. Date: Sep. 22, 2016

(54) METHODS AND MATERIALS TO TREAT JUNCTIONAL AND PELVIC HEMORRHAGE

(71) Applicants: Gregory T. ZUGATES, Chelmsford, MA (US); Adam Rago, Falmouth, MA (US); Toby Freyman, Waltham, MA (US); Upma Sharma, Somerville, MA (US); Rany Busold, Medford, MA (US); John Caulkins, Watertown, MA (US); Parisa Zamiri, Brookline, MA (US); Jennifer Mortensen, Somerville, MA (US); David King, Cambridge, MA (US)

(72) Inventors: Gregory T. ZUGATES, Chelmsford, MA (US); Adam Rago, Falmouth, MA (US); Toby Freyman, Waltham, MA (US); Upma Sharma, Somerville, MA (US); Rany Busold, Medford, MA (US); John Caulkins, Watertown, MA (US); Parisa Zamiri, Brookline, MA (US); Jennifer Mortensen, Somerville, MA (US); David King, Cambridge, MA (US)

(21) Appl. No.: 14/660,790

(22) Filed: Mar. 17, 2015

(51) Int. Cl. A61L 24/06; A61B 17/00

(52) U.S. Cl. CPC A61L 24/06; A61B 17/0057; A61B 2017/00654

(57) ABSTRACT

Systems and methods for the treatment of pelvic and junctional hemorrhage are provided, which utilize in situ forming polymer gels and foams to apply pressure to sites of hemorrhage due to penetrating or blunt injury, among other things.

### CROSS-REFERENCE TO RELATED APPLICATIONS

This application claims the benefit of priority under 35 U.S.C. §119(e) to U.S. Provisional Patent Application No. 61/954,142 by Zugates et. al titled “Methods and Materials to treat Junctional Hemorrhage” and filed Mar. 17, 2014. The foregoing application is incorporated by reference herein in its entirety and for all purposes.

### FIELD OF THE INVENTION

The present invention relates to medical devices and methods, and specifically concerns in situ forming polymer foams for use in the treatment of hemorrhage.

### BACKGROUND

In medicine, hemorrhage is frequently treated by the application of circumferential pressure to sites of bleeding. However, such pressure cannot be applied to bleeding from external, non-compressible wounds located at interfaces between the extremities and the torso (“junctional hemorrhage”) or from wounds in the pelvis. The present invention relates to methods and materials that can be used to stop or reduce such junctional and pelvic hemorrhage. Clinical sites of interest for this invention include the groin (iliac and femoral bleeding), axilla/clavicle region (brachial and subclavian arterial injuries), and the neck (carotid bleeding).

Injuries leading to hemorrhage are often classified as resulting from either penetrating or blunt trauma. Penetrating trauma is characterized by the presence of a foreign object penetrating the skin leading to injury; gunshot wounds are a common example. Alternatively, blunt trauma is typically caused by a high force impact, resulting in internal trauma without a distinct open injury or wound track. As an example, pelvic fractures resulting from high force, motor vehicle crashes can lead to severe internal trauma and hemorrhage.

### SUMMARY OF THE INVENTION

This invention, in its various aspects, utilizes in situ forming foam formulations to treat junctional and pelvic hemorrhage arising from both penetrating and blunt injuries.

For treatment of penetrating junctional or pelvic wounds, this invention allows liquid formulation to be deeply applied directly into the wound area, while still allowing the formulation to conform to the shape of the wounded area. This invention further allows previously “non-compressible” wound areas to be made compressible due to deep material penetration and a continuous structure. In one aspect of the present invention, in-situ foaming or gelling systems are combined with a device/material that assists in the application of the foam or gel, mediates its transport to the wound area, maintains its localization to the wound area, and/or enhances its effectiveness as a hemostatic agent. In another aspect of the present invention, pre-formed foams and materials are applied to a wound area to provide hemostasis.

Alternatively, for the treatment of blunt wounds at the junction, the present invention allows the liquid formulation to be applied near or adjacent to injuries. Doing so enables a “non-compressible” blunt injury to be compressed over a large surface area, which may be particularly advantageous in the treatment of diffuse bleeding. Alternatively or additionally, for treatment of severe bleeding in the pelvis, the present invention allows the liquid formulation to be applied through a small incision away from the injury. The resulting foam expands to fill a potential space, thereby making contact with and providing pressure to injured tissues.

More specifically, in one aspect, the present invention relates to a method of treating a junctional or pelvic hemorrhage in a patient that includes a step of depositing, at or near the hemorrhage, a fluid that is able to (i.e. is configured to) form a foam or a gel which applies pressure to the hemorrhage. The step of depositing the fluid may involve mixing a first fluid comprising a first reagent with a gas and/or a second fluid comprising a second reagent that interacts with the first reagent and/or with a bodily fluid to contribute to foam/gel formation. Alternatively or additionally, the step of depositing the fluid includes depositing the fluid in a penetrating wound, thereby applying pressure to at least part of the penetrating wound. The method may also include applying a compressive force to the foam/gel and/or the penetrating wound, for instance manually or by means of a covering (e.g. a bandage, sling, tourniquet, adhesive pad, mesh, etc.) that is placed about a portion of the patient's body. In other cases, the fluid is deposited within the preperitoneal space, in which case the foam or gel will expand into the retroperitoneal space.

In another aspect, the present invention relates to a method of treating junctional or pelvic hemorrhage that includes inserting a bag into the patient's body, then disposing a fluid that forms a gel or foam within the bag, thereby expanding the bag within the patient's body. The bag can include a hemostatic agent and it may include one or more structural features to facilitate introduction, application of pressure and/or removal. For instance, the bag may include adjacent first and second regions characterized by different cross-sectional areas, multiple lobes or fingers, and/or a tether that can be pulled (i.e. tension can be applied to it) to remove the bag. The bag can be inserted into the body at the same time it is filled with fluid in some cases. Alternatively or additionally, the bag and/or the foam or gel are biodegradable.

In yet another aspect, the present invention relates to a method of treating pelvic or junctional hemorrhage by disposing a fluid configured to form a foam within the patient's body such that the resulting foam applies pressure to the junctional or pelvic hemorrhage. The fluid is formed by mixing a first composition comprising a multifunctional isocyanate and a second composition including a polyol comprising up to 50 weight percent polyethylene oxide, up to 10 pphp of an amine catalyst, and up to 20 pphp water.

### DRAWINGS

Non-limiting embodiments of the present invention are described by way of example with reference to the accompanying figures, which are schematic and are not intended to be drawn to scale. Throughout the figures, each identical or nearly identical component is typically represented by a single numeral. For purposes of clarity, not every component is labeled in every figure, nor is every component of each embodiment of the disclosure shown where illustration is not necessary to those of ordinary skill in the art to understand the disclosure. In the figures:

FIG. 1 depicts a strap used to resist upward foam expansion.

FIG. 2 depicts a two-part foam system to seal and fill a wound area.

FIG. 3 depicts a retrieval rope used to recover hemostatic bag.

FIG. 4 depicts the delivery of a foaming formulation following gas insufflation of a wound.

FIG. 5 depicts the use of a foam fill a wound after sealing with a balloon.

FIG. 6 depicts the refraction of a delivery catheter to precisely control foam delivery.

FIG. 7 depicts a pre-formed foam fitted around a coiled spring.

FIG. 8A-B shows views of a foam according to the present invention deployed in a swine pelvic hemorrhage model.

FIG. 9A-B shows the foams removed intact from the swine model.

FIG. 10A-B shows bags according to certain embodiments of the present invention.

### DETAILED DESCRIPTION OF PREFERRED EMBODIMENTS

In the devices and methods of the present invention, foaming or gelling materials are delivered to a wound area, body cavity or potential space, where they can contribute to hemostasis by applying direct (i.e. by the foam or gel itself) or indirect (e.g. by means of a tissue or organ which to which pressure is applied by the foam or gel) pressure to sites of bleeding, by delivering hemostatic agents to sites of bleeding, and/or by excluding space into which blood would otherwise flow. The foaming and gelling materials of the present invention may hereafter be referred to as “formulations.” These materials may reduce or stop bleeding by exerting pressure, reducing the available space for blood to pool, enhancing coagulation, and/or abutting the wound surface to provide a seal. As used herein, a material is said to “foam” or “gel” in that it undergoes a chemical and/or physical change that results in the formation of foam, a gel, a semi-solid, or a more viscous fluid. As used herein, foaming or gelling materials are described as being “injected,” “deposited,” “applied,” delivered” and the like synonymously to mean that the foaming or gelling materials are placed at a target location on the patient's body using any suitable means. As used herein, “wound surface,” “wound area,” or “wound bed” refers to any wound or injury (a) located at or near an area on the body where an extremity such as the head, arm(s), or leg(s) meets the torso of the body, (b) within or proximate to the pelvis, or (c) in regions surrounding bony anatomy. As used herein, a material is described as a “fluid” if it is flowable, as is the case with, for example, fluid, semi-solid, and viscous materials.

The type of delivery device used to deliver the foams and gels of the present invention will depend upon, inter alia, the viscosity of the foaming or gelling material fluids. For example, some embodiments make use of a syringe needle, catheter, hand-powered syringe-assist, pneumatic pressure pump, or other similar device. If the system is comprised of multiple fluids, a multi-barrel syringe is preferably used for simultaneous injection of the fluids. To ensure adequate mixing, it is preferred to use a mixing nozzle, and more preferably a static mixing nozzle, mounted onto the multi-barrel syringe cartridge. In other embodiments, multiple fluids are combined just prior to delivery. Alternatively, multiple fluids can be delivered sequentially to the wound area. In some embodiments the fluids are delivered directly to the wound area, whereas in others the formulation delivery site may be in a location near or adjacent to the wound, rather than in the wound itself. In some embodiments, the fluids are injected as a single bolus, whereas in others the fluids are metered by someone skilled in the art until the appropriate amount of fluid has been delivered and/or until hemorrhage control has been achieved.

Exemplary delivery systems and methods which are useful in conjunction with the formulations and methods of the present invention are disclosed in U.S. patent application Ser. No. 14/211,469 (pre-grant publication no. US 20140228745 A1) by Sharma, et al. titled “Delivery System for In Situ Forming Foams and Methods of Using the Same,” which is incorporated by reference herein for all purposes.

The foams as used herein can be any suitable foam formed in-situ from a one, two, or multi-part formulation as described U.S. application Ser. No. 13/209,020, filed Aug. 12, 2011 and titled “In-situ Forming Hemostatic Foam Implants,” which is a continuation-in-part of U.S. application Ser. No. 12/862,362, filed Aug. 24, 2010 and titled “Systems and Methods Relating to Polymer Foams,” which claims priority to U.S. Provisional Patent Application Ser. No. 61/236,314 filed Aug. 24, 2009, titled “Systems and Methods Relating to Polymer Foams,” each of which are incorporated by reference herein for all purposes. Also incorporated by reference is the commonly-assigned U.S. patent application entitled “In-situ Forming Foams with Outer Layer,” filed concurrently herewith and naming Freyman et al. as in inventors. A preferred embodiment is a polyurethane foam formed in-situ from a one-part formulation consisting of an isocyanate-functionalized pre-polymer. This pre-polymer system could additionally contain multiple polymer species, catalysts, surfactants, chain extenders, crosslinkers, pore openers, fillers, plasticizers, and diluents. Gels as used herein can be generated by cross-linking suitable polyols with multifunctional isocyanates. Polyols suitable for use in such embodiments include polyether- and polybutadiene-based polyols. Polyols of particular interest include polypropylene glycol (PPG) and polyethylene glycol (PEG), as well as random and block copolymers thereof. Also suitable for use are polycarbonates, polybutadienes, and polyesters. The fluid foams or gels by the interaction between the pre-polymer fluids that are delivered simultaneously or sequentially, or by interaction with an aqueous environment (e.g., blood, water, and/or saline) upon or after delivery. Both foams and gels cross-link in-situ. In contrast to gels, foams are characterized by the presence of a pore structure in the crosslinked structure due to the generation of gas during the in-situ reaction of from the entrainment of gas in the initial injected formulation.

Any of the embodiments of the present invention may be formulated to be radiopaque, fluorescent, or otherwise visible by imaging techniques known to those skilled in the art. Radiopacity may be imparted by incorporation of iodinated contrast materials, barium sulfate, metal particles such as tantalum or titanium, etc. The foams or gels formed in-situ may be bio-resorbable, biocompatible, or non-absorbable, thereby reducing the quantity of foam which must be removed from the wound area following treatment or, in some cases, eliminating the need for removal of foam or gel material from the wound site altogether. In some embodiments, biodegradability is achieved by use of hydrolytically or enzymatically degradable polymers such as polyglycolic acid (PGA), polylactic acid (PLA), PLGA, polycaprolactone (PCL), polydioxanone, polyanhydrides, polyorthoesters, or various random and block copolymers. In other cases, the foam or gel is degraded by the application of an agent or stimulus, such as light, heat, ultrasound, an exogenous enzyme, etc. In still other, related cases, the foaming or gelling reactions are pressure dependent, such that the volume of the foam or gel does not significantly increase once the pressure in the wound area exceeds a predetermined threshold.

In some embodiments of the inventions, the foaming or gelling materials can further comprise agents known to enhance the endogenous blood coagulation process such as natural clotting factors (e.g., thrombin, fibrinogen/fibrin, factor X/Xa), chitin/chitosan, cellulose derivatives, alginate, gelatin, zeolites, and water-absorbent materials. These coagulation agents may be dissolved or suspended in the formulation or added as a separate phase at the time of delivery.

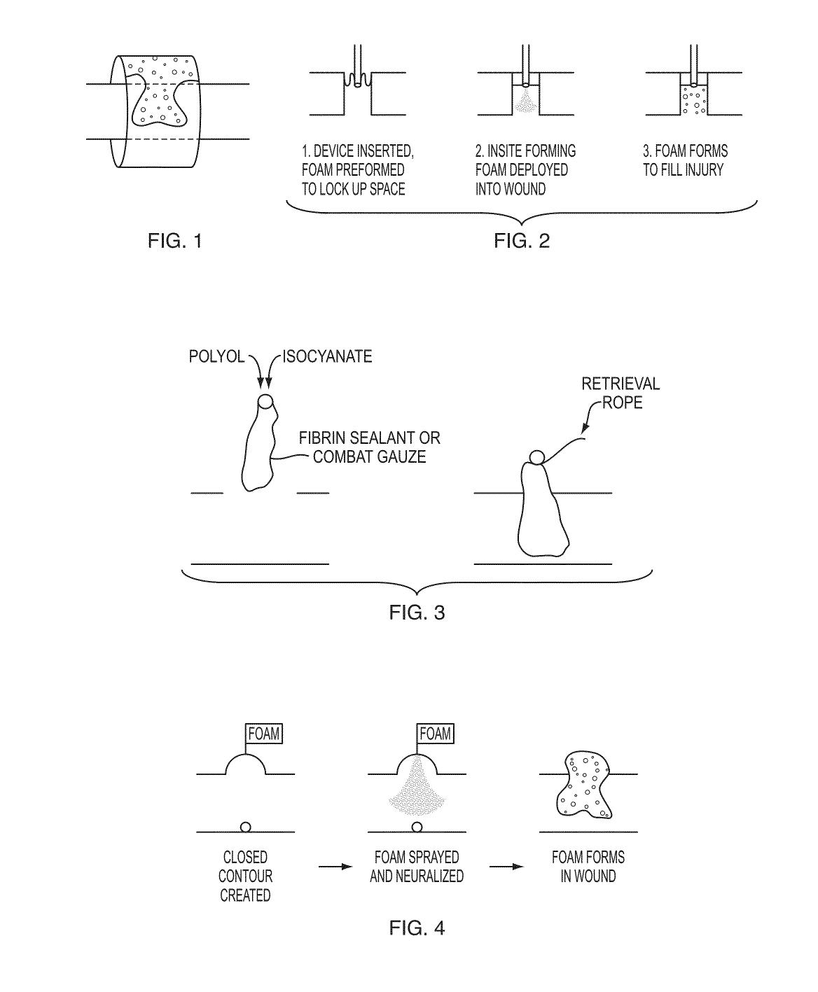

Foaming or gelling materials are, in some cases, combined with a device or material that provides a cover, seal or the like at the wound surface of penetrating injuries. The function of this seal is to prevent foam or gel movement out of the wound bed so that it may more fully penetrate bleeding sites. Given that the wound may be irregular and variable across injuries, the covering material can be applied as a wide sheet or made to be conformable to different wound geometries. In various embodiments, the covering 100 is a strap, adhesive pad, porous mesh or tourniquet that can be placed around the torso and/or extremities to provide a seal and promote directional expansion of the foam, as shown in FIG. 1. In some cases, the covering 100 is a structure adapted for use in the treatment of junctional or pelvic hemorrhage, such as a junctional tourniquet, pelvic binder or the like. The covering 100 may contain a port to facilitate fluid/formulation delivery, or it can be made of any suitable polymeric, metallic or composite material that is configured for delivery of the foaming or gelling materials therethrough. In some cases, the covering can be punctured by a needle or tube for such delivery.

In another embodiment, the foams or gels of the present invention are deployed into the wound (e.g., in penetrating wounds) and manual compression is applied thereto. Unlike conventional hemostatic products, the foams and gels of the present invention are formed as deeply penetrating structures that are continuous (monolithic) up to the wound surface, so that the compression force applied to the exterior of the foam may be transmitted deep into the wound.

In another embodiment, the foams or gels of the present invention are deployed adjacent or near to injuries (e.g. in treatment of blunt injuries). In some cases the foams or gels are positioned so they can fill a potential space created by expansion between tissue planes or are applied against an applied barrier such as covering 100, described above. As another non-limiting example, blunt trauma to the pelvis can often result in displacement of the pelvic ring. Hemorrhage resulting from these injuries is often severe and can be diffuse due to in bleeding from the venous plexuses. Typically, this bleeding occurs into the retroperitoneal space, and as a closed space, this bleeding has the ability to naturally tamponade. However, displacement of the pelvic ring commonly leads to injury of the retroperitoneal space, disrupting this closed space. In humans, the volume of the pelvic space following injury, while highly variable, can be as much as four liters, such that uncontrolled bleeding into the pelvic space can lead to exsanguination. This invention allows for this “non-compressible” hemorrhage to be compressed over a wide surface area by applying the in-situ foam or gel near or adjacent to the injury. For example, the in-situ foam or gel may be delivered into the retroperitoneal space, thereby applying pressure over a large surface area. This approach is similar to the current techniques for packing the retropertitoneal space with the advantage that using an in-situ foam or gel will provide better conformal contact, and thereby compression of bleeding surfaces, than pre-formed laparotomy pads. Alternatively, the in-situ foam or gel may be applied in the pre-peritoneal space (the space adjacent to the injury), whereupon it expands throughout the preperitoneal and retroperitoneal spaces potential space is pressurized leading to compression of the injuries. This concept has been tested by the inventors in a swine model, as set forth in Example 1, below.

In one embodiment, the invention makes use of a two-part system in which a formulation is delivered to create a seal over the wound area. By deploying the formulation as a fluid, the formulation material conforms to the irregular wound opening and may expand as a foam to seal the opening. In other embodiments, the formulation forms a gel. This gelling material is deployed as a liquid polymer formulation that transitions into a non-expanding gel. The gelling material may be comprised of a polyol blended with a multi-functional isocyanate. Catalysts such as DABCO or zinc octoate may be added to adjust the gelling time, which preferably occurs in less than ten minutes, more preferably in less than 5 minutes, and most preferably in less than two minutes. In some cases, the formulation release a volume of gas such as CO₂ during its expansion and solidification, which gas contributes to an increase of local pressure around the implant and augmenting the tamponade effect of the foam or gel.

The sealing foams and gels of the present invention may be held in place manually, by virtue of its expanded fit into the wound bed, or by its placement in a virtual or potential space or compartment within the body. In other embodiments, the sealing foam or gel may be held in place by a bandage, strap, belt, adhesive mechanism, or any other device known in the art. Where the sealing material is a foam, the sealing foam may be used as a means to apply compression to the injury through the expansion thereof. In yet other embodiments, the sealing foam or gel can be designed to bind or adhere to the perimeter of the wound area by incorporation of agents that specifically bind injured tissues (e.g., peptides, sugars) or inclusion of tissue adhesives (e.g., cyanoacrylates, polyisocyanates). In additional embodiments, agents or ligands that bind uninjured or healthy tissue can also be incorporated to further enhance the seal beyond the wound perimeter and increase the strength and resistance of the sealing foam or gel.

In any embodiment of the invention, once a sealing foam or gel is in place into, over, or around a wound, a second formulation can be delivered underneath the previously-delivered foam or gel (i.e., between the previously-delivered foam or gel and the wound tissue) as shown in FIG. 2. It is preferable that this second formulation has a low viscosity to aid in its distribution and penetration in the wound cavity. In some embodiments, the second formulation is a foam. As the second formulation expands and rises as a foam, its expansion will be limited by the previously delivered foam or gel, thus applying a force into the wound. Alternatively, the second formulation can be delivered alongside the first formulation, or the formulation can include a mixture of (a) a dense one-part material that reacts with blood in the wound space, and (b) a highly expandable material that provides pressure throughout the wound area. This arrangement may achieve more complete filling of spaces and/or potential spaces in and around the wound area.

More generally, any number of formulations or components with different functional characteristics (e.g. stiffness, expansion, compliance) can be used to treat junctional hemorrhage. For instance, in some embodiments a pre-formed gel or foam may be first placed within a cavity to act as a mechanical blockade to limit the path of the foam, to provide a surface or structure against which a second foam can expand (e.g., when administering foam into the retroperitoneal or pre-peritoneal space a lap pad may first be placed). The in-situ foam or gel can then be filled into the cavity or potential space.

In other embodiments, the second formulation is comprised of gelling material. This material could be a polymer mixture that gels upon reaction of two or more components to form a crosslinked network. Alternatively, the gelling mixture can contain a blood gelling agent, which physically crosslinks blood, for example an agent that electrostatically binds with red blood cell membranes.

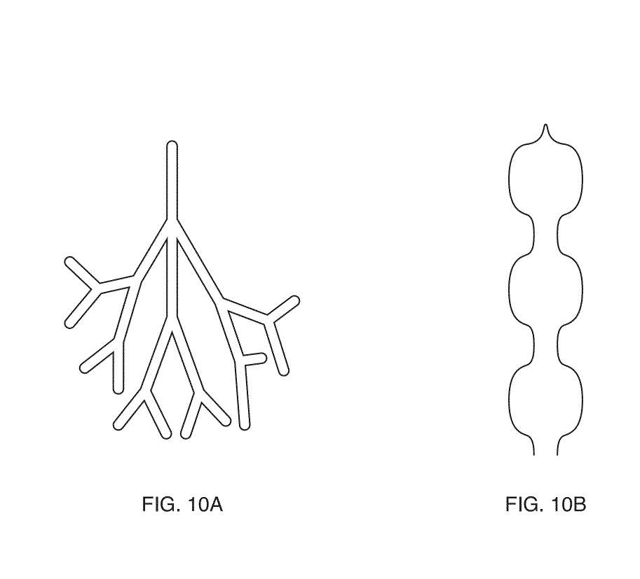

In another group of embodiments, an in-situ forming foam can be used to drive a preformed, solid hemostatic material into a wound cavity to improve its depth of penetration and proximity to the bleeding sources. In one example, a flexible bag- or balloon-like structure is filled with a formulation, driving the bag into the wound area where it can contact and/or apply pressure. As used herein, “bag” and “balloon” may be used interchangeably to describe any suitable compliant, semi-compliant, or non-compliant structure having an internal open space. The bag, which is optionally (but not necessarily) fabricated from hemostatic agent, or from a material that incorporates a hemostatic agent or has hemostatic properties, is placed into the wound. A liquid foaming or gelling formulation can be delivered into the hemostatic bag and held in place by physical compression. In certain embodiments, the foam is introduced into the structure in one step or multiple steps before, concurrently with, or after introduction of the bag into the wound. The bag expands with the foam such that its surfaces are pressed into the wound. Optionally, the bag can be porous so that some foam expands beyond the bag wall to adhere to the wounded tissue. The pores may be numbered and arranged to best promote hemostasis. For example, there may only be one pore at the part of the bag deepest in the wound to anchor the bag in the wound area. In other embodiments, there may be a plurality of pores located uniformly around the bag, or the pores may actually be slits placed at desired locations within the bag wall. The bag 200 is generally characterized by a fully expanded shape, which shape optionally includes one or more constricted (i.e. smaller cross-sectional area) regions 205 relative to at least one larger cross-sectional area region, and/or a plurality of lobes, one or more fingers 210, an hourglass structure, or another regular or irregular structure having varying cross-sectional areas as shown in FIG. 10A-B.

The bag- or balloon-like hemostatic structure is a non-limiting example of a solid surface used as a sealing or hemostatic structure in conjunction with the present invention. In other embodiments, structures such as gauze, sheets, powders, particles, or any number of other forms may be used. The structure may be biostable or bioresorbable (i.e., formed from a material that resorbs, absorbs, erodes, or is otherwise eliminated from the body). The structure optionally comprises a means for removal or retrieval, such as a retrieval rope or suture as depicted in FIG. 3. Other elements of a foam or gel that may facilitate removal include a lubricious membrane or coating, a perforated region or a region which is predisposed to tear or fracture, permitting the foam to be removed in multiple pieces, etc. Removal can also be facilitated though the incorporation of materials or chemistries that increase the softness/compliance and resiliency of the foam or gel and/or a foam or gel with reticulated characteristics, such that the foam or gel can be removed through a small midline.

In another embodiment of the invention, a wound can be sealed off by use of a structure such as an adhesive pad, suction-cup based device or other device, which creates an airtight seal over the wound. A space is thereafter created between the structure and the wound tissue by insufflation. Using delivery mechanisms previously described, a formulation of the present invention can then be delivered within this closed but insufflated space to penetrate the wound site. Alternatively, the formulation can be aerosolized and deployed as small droplets that are delivered into the insufflated space to cover the wounded surface, as shown in FIG. 4.

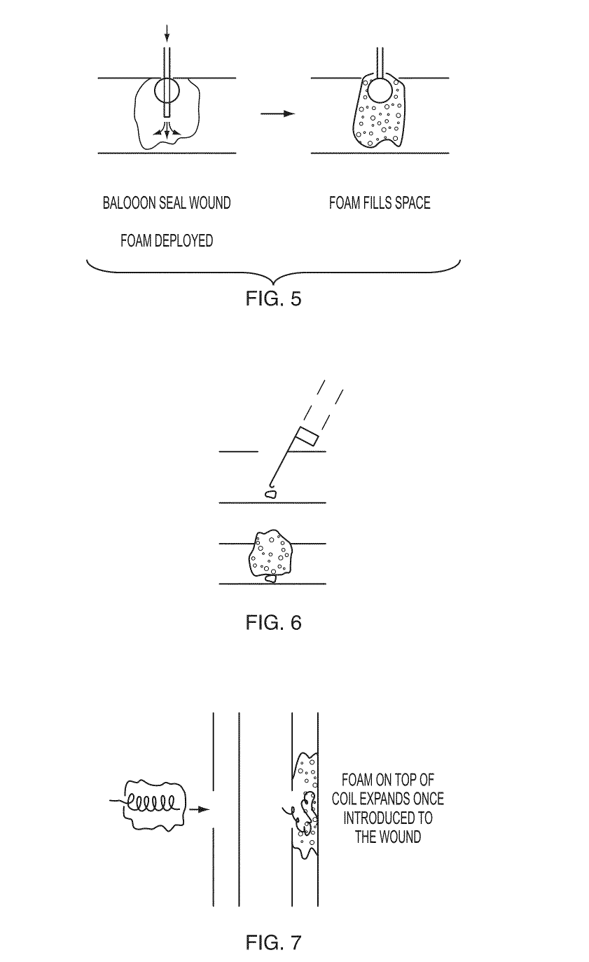

In another embodiment, a wound surface can be sealed by the application of an expandable structure such as an inflatable balloon, pre-formed foam, or the like. The expandable structure optionally comprises a shaft or passageway through which a user may deliver liquid foam formulation into the wound area after sealing of the wound surface, as depicted in FIG. 5. As described above, the balloon or foam may contain tissue adhesives/ligands or can be made porous to allow some liquid foam formulation to permeate through and contact the wound margins.

In certain embodiments, a long, thin needle, catheter, tube or applicator (referred to generally as “applicators”) may be used as a delivery device to deliver the liquid formulation. Proximity of the formulation to the injured tissues is improved by inserting the needle deep within the wound. By pulling back on the delivery device as the formulation material is expelled, the material can foam at various tissue depths. In a similar fashion, the delivery applicator can be designed to retract at predetermined speeds as the liquid formulation is delivered, so that the depths of foam placement are more precisely controlled as shown in FIG. 6. A preferred foam for use in such embodiments would have a low initial viscosity and rapid foaming kinetics so that it spreads quickly and sets in place rapidly.

In certain embodiments, a long, thin tube or catheter with a water-soluble outer wall is used as a delivery device. The delivery device is inserted into the wound opening and advanced towards the wound site. When it contacts blood or water in the surrounding tissues, the outer wall dissolves to release the formulation contained within the delivery device. The formulation may be a two-part polyurethane that is mixed prior to loading or during delivery. Alternatively, a one-part polyisocyanate can be used that foams on contact with blood or water. In another embodiment, the liquid formulation may not foam, but rather form a crosslinked gel after being delivered to the targeting bleeding sites.

While the embodiments above describe in-situ foams and gels, other aspects of the invention use pre-formed materials such as foams, gels, deformable clays, meshes, or particles to stop or reduce bleeding. As used herein, a “pre-formed” material is prepared outside of the body prior to insertion, delivery, or installation into or on the wounded area. The materials can be shaped by the user's hands, or may be shaped by a mold. They may be designed to be thin strips or sheets, and may be perforated to optimize wound packing All of the considerations mentioned above for in-situ foaming systems apply here, including the use of pro-coagulants, tissue-binding ligands and adhesives, and biodegradable polymers.

In some embodiments, pre-formed materials are constructed with pro-coagulant agents. The agents can be added to the formulation prior to foaming, or applied to the surface of the pre-formed foam. After the foam sets, it can be used to apply pressure to the wound, or can be used to pack the wound to achieve hemostasis. In other embodiments, a semi-plastic or amorphous solid that is deformable can be used as the pre-formed material. The deformability of the material allows it to conform to the wound tissue surfaces upon pressure-based application. The material may cause hemostasis by surface activation of blood clotting factors, absorbing fluids to concentrate coagulation factors, filling a void space, or providing a tamponade effect.

In some embodiments, granules or particles of an absorbent or superabsorbent material can injected or pressed into the wound. Absorption of body fluids may cause a local increase in the concentration of coagulation factors and accelerate hemostasis.

In some embodiments, pre-formed materials are delivered into the wound via a tube, catheter, or other delivery means. The material can be compressed and collapsed down to fit into the tube lumen and by retraction or pushing, the material can be withdrawn or expelled from the tube. As the pre-formed material becomes decompressed, it may expand to conform to the walls of the wound area. Furthermore, the tube walls could be made of a water-soluble material such that the material is released when the tube contacts body fluids due to dissolution of the tube wall.

In some embodiments, a pre-formed material is fitted around a coiled tube or spring and inserted into the wound as shown in FIG. 7. The coil provides maneuverability and trackability to deliver the material deep into the tissues. Water within the tissue causes the material to expand or swell, which thereby causes apposition with the bleeding vessels. In addition to a single coil, multiple foam-coils could be introduced into the wound from the same or different entry sites.

Finally, the principles and embodiments of the present invention can be further understood by means of the following non-limiting examples:

### Example 1

### Swine Pelvic Hemorrhage Model

A pilot study demonstrated that a self-expanding two-part polyurethane foam could successfully distribute throughout the pre-peritoneal space. This study was conducted in a swine cadaver following a pre-clinical experiment in a severe grade V liver injury model. Foam formulation AM1276 was prepared according to TR-0130. The pre-peritoneal space was accessed using a 5-cm incision just below the umbilicus, and foam was deployed into the space at an initial volume of 100 mL. The instrumentation incision was sealed after deployment using a penetrating towel clamp.

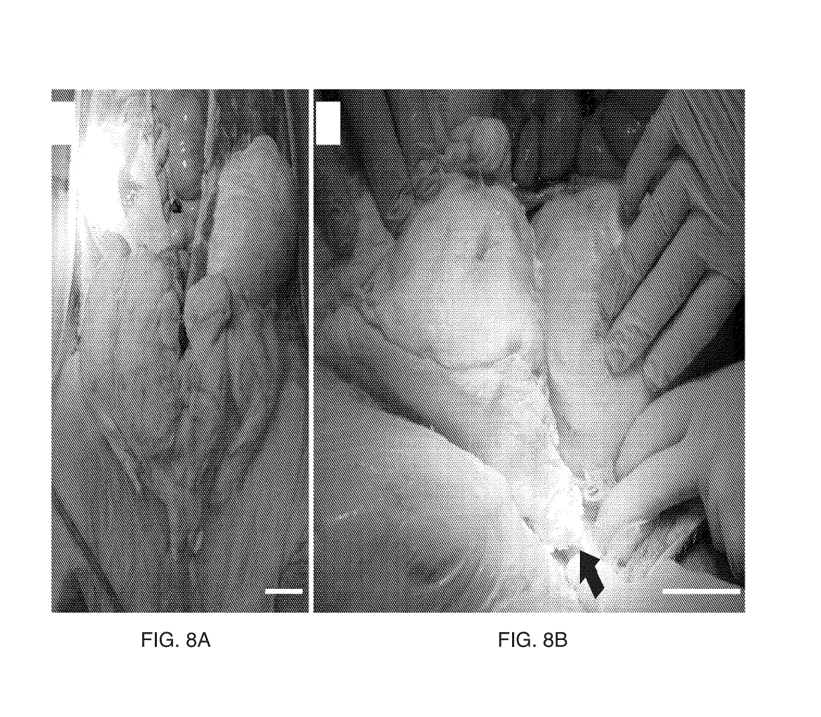

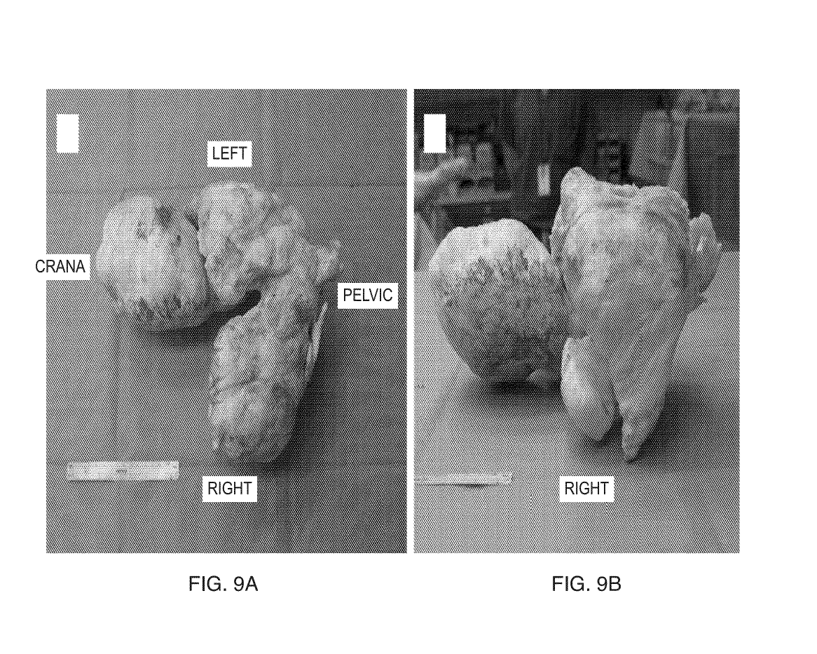

As shown in FIGS. 8A-B, the foam 1 distributed throughout the pre-peritoneal space, conforming to local anatomy. Foam was observed in contact with the region containing the iliac arteries. It is expected that this foam distribution could provide hemostasis in pelvic hemorrhage. The extent of the foam expansion within the preperitoneal space is further illustrated by top and side views of explanted foams as shown in FIGS. 9A-B.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. The term “substantially linear” is used in this specification to describe an arrangement that is at least partially “straightened out,” for instance, as a wire, ribbon, fiber or other elongated, flexible body may become straightened out when placed under tension. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method of treating a pelvic or junctional hemorrhage in a patient in need thereof, the method comprising:
depositing, at or near the pelvic or junctional hemorrhage, a fluid configured to form a foam or gel, thereby applying pressure to the pelvic or junctional hemorrhage.

2. The method of claim 1, wherein the step of depositing the fluid configured to form a foam or gel includes mixing a first fluid comprising a first reagent with at least one of a gas and a second fluid comprising a second reagent that interacts with the first reagent or a bodily fluid to contribute to the formation of the foam or gel.

3. The method of claim 1, wherein the step of depositing the fluid configured to form a foam or a gel includes depositing the fluid into a penetrating wound, thereby applying pressure to at least a portion of the penetrating wound.

4. The method of claim 3, further comprising the step of applying a compressive force to the foam or gel and the penetrating wound.

5. The method of claim 4, wherein the step of applying compressive force includes manually compressing the foam or gel.

6. The method of claim 4, wherein the step of applying compressive force includes contacting at least one of the foam or gel and the penetrating wound with a covering placed about a portion of the body of a patient.

7. The method of claim 1, wherein the step of depositing the fluid at or near the site of the pelvic or junctional hemorrhage includes depositing the fluid within the pre-peritoneal space, wherein the foam or gel expands into the retroperitoneal space.

8. The method of claim 1, further comprising the step of forming the fluid by mixing a first composition comprising a multifunctional isocyanate with a second composition including a polyol comprising up to 50 weight percent polyethylene oxide, up to 10 parts per hundred polyol (“pphp”) of an amine catalyst, and up to 20 pphp water.

9. The method of claim 8, wherein the fluid is configured to form a foam with a rise time of less than 150 seconds.

10. A method of treating a junctional or pelvic hemorrhage in a patient in need thereof, comprising the steps of:
inserting into the body of the patient at least a portion of a bag; and
disposing, within the bag, a fluid configured to form a foam or gel, thereby expanding the bag within the body of the patient.

11. The method of claim 9, wherein the bag includes a hemostatic agent.

12. The method of claim 9, wherein the bag includes adjacent first and second regions characterized by different cross-sectional areas.

13. The method of claim 9, wherein the bag comprises a plurality of lobes or fingers.

14. The method of claim 9, wherein the steps of inserting a portion of the bag into the body of the patient and disposing the fluid within the bag performed concurrently.

15. The method of claim 9, wherein the bag includes a tether configured to permit a user to remove the bag from the body of the patient by applying tension to the tether.

16. The method of claim 9, wherein at least one of the bag and the foam or gel is biodegradable.

17. The method of claim 9, further comprising the step of forming the fluid by mixing a first composition comprising a multifunctional isocyanate and a second composition including a polyol comprising up to 50 weight percent polyethylene oxide, up to 10 pphp of an amine catalyst, and up to 20 pphp water.

18. The method of claim 17, wherein the fluid is configured to form a foam with a rise time of less than 150 seconds.

19. A method of treating a junctional or pelvic hemorrhage in a patient in need thereof, comprising the steps of:
mixing a first composition comprising a multifunctional isocyanate with a second composition including a polyol comprising up to 50 weight percent polyethylene oxide, up to 10 pphp of an amine catalyst, and up to 20 pphp water, thereby forming a fluid configured to form a foam; and
disposing the fluid within the body of a patient such that the resulting foam applies a pressure to the junctional or pelvic hemorrhage.
